(19) United States
(12) Patent Application Publication
(10) Pub. No.: US 2026/0137089 A1
(43) Pub. Date: May 21, 2026

(54) TALAROMYCES PURPUREOGENUS AND APPLICATION THEREOF IN CONTROL OF COTTON DISEASES

(71) Applicants: INSTITUTE OF COTTON RESEARCH OF CAAS, Anyang (CN); INSTITUTE OF WESTERN AGRICULTURE, CAAS, Changji Hui Autonomous Prefecture (CN)

(72) Inventors: Zili Feng, Anyang (CN); Heqin Zhu, Anyang (CN); Peng Li, Anyang (CN); Feng Wei, Anyang (CN); Hongjie Feng, Anyang (CN); Lihong Zhao, Anyang (CN); Yalin Zhang, Anyang (CN); Jinglong Zhou, Anyang (CN); Yong Song, Changji Hui Autonomous Prefecture (CN)

(21) Appl. No.: 19/412,734

(22) Filed: Dec. 8, 2025

Related U.S. Application Data
(63) Continuation of application No. PCT/CN2025/133471, filed on Nov. 7, 2025.

(30) Foreign Application Priority Data
Nov. 19, 2024 (CN) 202411657167.5

(51) Int. Cl. A01N 63/30; A01P 3/00; C12R 1/645

(52) U.S. Cl. CPC A01N 63/30; A01P 3/00; C12N 1/145; C12R 2001/645

(57) ABSTRACT

A Talaromyces purpureogenus and an application thereof in control of cotton diseases, a deposit number of the Talaromyces purpureogenus is CCTCCNO: M20242093. An application of the Talaromyces purpureogenus CEF642N is an application as a biocontrol agent for cotton Verticillium wilt. In the present disclosure, after analyzing the inhibitory effect of extract components of Talaromyces purpureogenus CEF642N on Verticillium dahliae, it is found that an extract of Talaromyces purpureogenus CEF642N has the effect of controlling Verticillium wilt and can be used to prepare a biocontrol agent, thereby providing a new biocontrol method for the control of Verticillium wilt.

### CROSS-REFERENCE TO RELATED APPLICATIONS

This application is a continuation of PCT/CN2025/133471, filed on Nov. 7, 2025 and claims priority of Chinese Patent Application No. 202411657167.5, filed on Nov. 19, 2024, the entire contents of which are incorporated herein by reference.

### TECHNICAL FIELD

The present disclosure relates to the technical field of biological control, and in particular to a Talaromyces purpureogenus and an application thereof in control of cotton diseases.

### BACKGROUND

Verticillium wilt is one of the most destructive fungal diseases in cotton growing areas around the world, causing severe yield losses and fiber quality degradation. Its main pathogenic factor is a soil-borne filamentous fungus, which can cause vascular wilting in many important crops. Biological control has attracted increasing attention due to the lack of effective chemical agents to eradicate Verticillium dahliae in soil. At present, the biological control microorganisms for controlling Verticillium wilt are mainly concentrated in Trichoderma, non-pathogenic Fusarium, Demodex flavus and Bacillus, Pseudomonas and Streptomyces.

Endophytic fungi colonize the intercellular and/or intracellular regions of healthy plant tissues and have a close symbiotic relationship with the host. These microorganisms produce antibiotics, enzymes, and volatile compounds to protect host plants thereof from other bacterial and fungal pathogens. Endophytic fungi are used as the most common microbial preparations against plant pathogens and are ubiquitous in all plant parts. Natural products and microorganisms have been used worldwide as biopesticides because natural products and microorganisms are accessible from the environment, are generally safe for non-target organisms including humans, the persistence of natural products and microorganisms in the environment is reduced, and natural products and microorganisms may be useful in organic agriculture. Talaromyces is widely distributed in soil, plants, sponges, and food. Recent findings have shown that Talaromyces is abundant in marine environments. The secondary metabolites of Talaromyces are rich in species, novel in structure and good in biological activity, which provides a basis for the development and application of endophytes.

Microbial volatile organic compounds (MVOCs) are small molecules belonging to different chemical classes, including alkenes, alcohols, ketones, organic acids, terpenes, benzenoids, and pyrazines. MVOCs can cause many changes in these microorganisms, such as vacuolation, fungal hyphal breakage, loss of intracellular components, regulation of metabolic and disease-causing genes, and expression of proteins important in host responses. Volatile organic compounds (VOCs) are considered as a promising and sustainable biocontrol strategy that can replace pesticides and fertilizers. Mycoparasitism or hyperparasitism is a parasitic interaction between one fungus and another, and serves as a direct biocontrol mechanism for controlling phytopathogenic fungi. Fungi can kill plant pathogens, protect plants from abiotic and biotic stresses, and reduce disease incidence and severity at the plant population level.

### SUMMARY

An objective of the present disclosure is to provide a Talaromyces purpureogenus and an application thereof in control of cotton diseases, that is, a Talaromyces purpureogenus CEF642N and an application thereof in control of cotton Verticillium wilt.

The present disclosure firstly provides a strain of Taralomyces purpureogenus CEF642N, which is deposited in the China Center for Type Culture Collection of Wuhan and Wuhan University in China on Sep. 26th, 2024, with a deposit number of CCTCCNO: M20242093.

In a further aspect of the present disclosure, an application of the Talaromyces purpureogenus CEF642N is provided, which is an application as a biocontrol agent for cotton Verticillium wilt.

In a further aspect of the present disclosure, the other application of the Talaromyces purpureogenus CEF642N is further provided, which is an application in preparing a product for inhibiting Verticillium dahliae.

In a further aspect of the present disclosure, a liquid formulation for controlling cotton Verticillium wilt is further provided, which is prepared by adding an extract of Talaromyces purpureogenus CEF642N into a solvent.

Further, the extract of the preparation, Talaromyces purpureogenus CEF642N, has a concentration of 300 μg/mL.

The present disclosure further provides a method for controlling cotton Verticillium wilt, which uses Talaromyces purpureogenus CEF642N as a biocontrol agent to control cotton Verticillium wilt.

Preferably, in the method, a concentration of the extract of Talaromyces purpureogenus CEF642N is 300 μg/mL.

In the present disclosure, after analyzing the inhibitory effect of extract components of Talaromyces purpureogenus CEF642N on Verticillium dahliae, it is found that the extract of Talaromyces purpureogenus CEF642N has the effect of controlling Verticillium wilt, and can be used for preparing biocontrol agents, thereby providing a new biocontrol method for the control of Verticillium wilt.

### BRIEF DESCRIPTION OF THE DRAWINGS

FIG. 1 shows effect of VOCs on an ultrastructure of Verticillium dahliae. (A1), (A2), and (A3) are scanning electron microscope (SEM) images of Verticillium dahliae cultured alone for 15 days at different magnifications; (B1), (B2), and (B3) are SEM images of Verticillium dahliae treated with 800 μL/L 1-octen-3-one for 15 days at different magnifications; and (C1), (C2), and (C3) are SEM images of Verticillium dahliae treated with total VOCs from CEF642N for 15 days at different magnifications.

FIG. 2A shows changes in intracellular structures of Verticillium dahliae after 15 days of treatment with VOCs from CEF642N, and shows a transmission electron micrograph (TEM) image of Verticillium dahliae in a control group, with CW indicating cell wall and M indicating mitochondria.

FIG. 2B shows a TEM image of Verticillium dahliae treated with 800 μL/L 1-octen-3-one, with CW indicating cell wall and LB indicating liposome.

FIG. 2C shows a TEM image of Verticillium dahliae treated with total VOCs from CEF642N via a double coating method, with CW indicating cell wall and LB indicating liposome.

FIG. 3 shows a temporal process of mycoparasitic effect of CEF642N on Vd076.

FIG. 4A shows an SEM image of CEF642N cultured alone for 15 days at 1000× magnification.

FIG. 4B shows an SEM image of Vd076 cultured alone for 15 days at 1000× magnification.

FIG. 4C shows an SEM image of CEF642N and Vd076 co-cultured in confrontation for 15 days at 1000× magnification.

FIG. 4D shows an SEM image of CEF642N and Vd076 co-cultured in confrontation for 15 days at 2000× magnification.

### DETAILED DESCRIPTION

A strain CEF642N of the present disclosure was isolated from healthy cotton roots in Anyang City, Henan Province. CEF642N was inoculated on potato dextrose agar (PDA) medium, and a sterile coverslip was obliquely inserted next to the PDA medium. After 10 days, hyphae were covered with a coverslip, the coverslip was removed, and conidia were observed under a fluorescence microscope (Nikon, ECLIPSE80i). CEF642N was cultured in PDA medium for 15 days, cut into 2 cm×2 cm pieces, and placed on a metal table coated with conductive adhesive. CEF642N was rapidly frozen in liquid nitrogen for 1-2 minutes, followed by sublimation for 15 minutes and gold sputtering for 1 minute. The morphology of hyphae was observed by an SEM (SU3500) in a state filled with liquid nitrogen. A phylogenetic tree of CEF642N was constructed using a neighbor-joining method in MEGA5 software based on an internal transcribed spacer (ITS) sequence of CEF642N.

Confrontation culture: CEF642N (5 mm in diameter) was inoculated at one end of the PDA medium, and Vd076 (5 mm in diameter) was inoculated at the other end. Treatment groups were inoculated with Vd076 and CEF642N at a position 20 mm away from centers of Petri dishes, with the same diameter. The control groups were Petri dishes inoculated only with Vd076 and CEF642N. The colony diameter was measured by the cross-cross method after 3 days of culture at 25° C. The data were recorded, and a bacteriostasis rate was determined, with a calculation formula of bacteriostasis rate (%)=[(control group colony diameter-5mm)−(treatment group colony diameter-5mm)]/(control group colony diameter-5mm)×100.

Double Petri dish culture: CEF642N (diameter 5 mm) was inoculated in a middle of a Petri dish including PDA medium, and Vd076 (diameter 5 mm) was inoculated in a middle of the other Petri dish including PDA medium. The two Petri dishes were inverted and sealed with sealing membranes, with Vd076 on a top and CEF642N on a bottom. The medium inoculated with Vd076 alone was used as the control. Cultivation was performed at 25° C., monitored and photographed. Colony diameters were measured by the criss-cross method. Data were collected, and inhibition rates were calculated.

The present disclosure will be described in detail below with reference to specific examples and accompanying drawings.

### Example 1: Antifungal Activity of Crude Extract

The crude extract was dissolved in dimethyl sulfoxide (DMSO), sterilized to remove miscellaneous bacteria using a 0.22 μm microporous filter membrane, and added into PDA media to achieve final concentrations of 300, 150, 75, 37.5, 18.75, and 0 μg/mL, with a constant DMSO concentration of 2% in all media. Positive control groups were treated with carbendazim at final concentrations of 2, 1, 0.5, 0.25, and 0.125μg/mL. Centers of the Petri dishes were inoculated with Vd076. Colony diameters were measured every 3 days, data were collected, and the inhibition rates were determined. GraphPad Prism 8 was used to calculate the 50% inhibitory concentration (EC50).

A fungal strain isolated from healthy cotton roots in this laboratory was found to have significant potential to inhibit the growth of Verticillium dahliae. CEF642N was shown to exert a strong inhibitory effect on Vd076 on the 15th day of confrontation culture, with an inhibition rate of 52.76%. On the 6th and 9th days of confrontation culture, when antagonistic fungus had no contact with the pathogenic fungus, Verticillium dahliae was observed to grow deviating from the center. It was speculated that CEF642N secreted organic substances with antifungal activity, which inhibited the growth of Vd076 through agar diffusion. In the inverted paired assay, it was found that the VOCs produced by CEF642N exhibited a significant inhibitory effect on Vd076 without any physical contact. CEF642N exerted a mycoparasitic effect on Vd076 on the 15th day of confrontation culture. This inhibitory effect is caused by multiple reasons, including VOCs, non-VOCs, antimicrobial peptides, cell wall-degrading enzymes, as well as competition for nutrients and space, since CEF642N and Vd076 coexist in the same Petri dish.

AntiSMASH was used to predict biosynthetic gene clusters of compounds in strain CEF642N, and 19 gene clusters were identified, including polyketide synthase (PKS), non-ribosomal peptide synthase (NRPS), and terpenes, etc. Several bioactive compounds with significant anticancer, antibacterial, enzyme inhibitory, and antimalarial activities were identified. Duklacillins are dimeric and heptacyclic fungal polyketides with significant biological activity. Duklacillins inhibit the production of adenosine triphosphate (ATP) through mitochondrial respiration, effectively inhibiting several tumor cell lines. Syringic acid is a farnesyltransferase inhibitor (FTI) with anticancer activity. To verify the genomic prediction results, the ethyl acetate extract of CEF642N was detected using the liquid chromatography-tandem mass spectrometry (LC-MS/MS) method. A total of 6414 compounds were found, of which 662 were in negative mode. The compound with the highest content was [m-H]-m/z 519.1889, followed by m/z 269.104 and m/z 425.1101.

To determine whether the compounds of strain CEF642N are related to antifungal activity, potato dextrose broth (PDB) cultures were extracted using ethyl acetate as the solvent. The results showed that CEF642N extract had different levels of inhibitory effect on the growth of Vd076. CEF642N extract was effective in inhibiting Verticillium dahliae at 150 μg/mL, resulting in smaller and malformed fungal colonies. At 300 μg/mL, the growth of Verticillium dahliae was completely inhibited. Consistent with the confrontation culture, different concentrations of the crude extract of CEF642N were added to Oxford cups. The results showed that the diffusion of non-volatile compounds produced by CEF642N was restricted to a specific range. However, CEF642N continued to grow in the Petri dishes, and during the confrontation culture, Verticillium dahliae exhibited off-center growth, which is consistent with this conclusion.

The mass spectra of GC-MS were compared with the NIST 2017 database. A total of 411 peaks were detected, and 331 VOCs were identified. 6 VOCs had relative peak areas greater than 0.5. These VOCs are 1,3-octadiene (retention time Rt=3.371 min), 3-octanone (Rt=11.296 min), 1-octen-3-one (Rt=12.629 min), n-hexanol (Rt=14.247 min), 1-octen-3-ol (Rt=14.247 min), and 2-octen-1-ol (Rt=20.96 min).

Over time, compared with the 6th-day group, 5 VOCs were upregulated, 46 VOCs were downregulated, and 280 VOCs remained unchanged in the 15th-day group. For the 63 VOCs with peak areas greater than 0.01, cluster heatmap analysis revealed three major clusters. In Cluster 1, the relative content of VOCs was higher in the 6th-day group than in the 15th-day group, while in Clusters 2 and 3, the relative content of VOCs was higher in the 15th-day group. Combined analysis of the volcano plot and cluster heatmap of VOCs in CEF642N showed that among the 331 annotated VOCs, the relative content of low-abundance VOCs was significantly higher on the 6th day than on the 15th day.

The proportion of major VOCs changed over time. In the 15th-day group, the top three VOCs were 1-octen-3-ol (21.87%), 2-octen-1-ol (21.23%), and 3-octanone (20.74%). In the 6th-day group, the top three were 2-octen-1-ol (28.54%), 1-octen-3-ol (19.19%), and 1-octen-3-one (11.81%). The relative percentage of 1-octen-3-one in the early stage of culture (6 days) was higher than that in the late stage (15 days), which may be attributed to the reduction of ketones to alcohols catalyzed by alcohol oxidoreductases. To demonstrate the potential biological effects of 6 VOCs, pure standards were purchased and the antagonistic activity thereof against Verticillium dahliae was studied.

### Example 2: Inhibitory Effect of VOCs from Talaromyces purpureogenus on Verticillium dahliae

1. Inhibitory Effect on Colony Growth of Verticillium dahliae

To determine the effect of volatile compounds on Verticillium dahliae, 20 mL of PDA medium was placed in a 70 mL Petri dish. Similar to the confrontation culture, Vd076 was inoculated on one side, and an Oxford cup with the same diameter, 20 mm away from the center of the Petri dish, was placed on the other side. Different volumes of trans- 2-octen-1-ol, 1-octen-3-ol, 3-octanone, and 1-octen-3-one were added to the Oxford cup. 1-octen-3-one was diluted with chromatographic methanol to concentrations ranging from 1.5625 to 3200 μL/L. The culture conditions and calculation of inhibition rate were consistent with those described previously.

After 15 days of culture on PDA medium, standards at different concentrations exhibited inhibitory effects on the hyphae of Verticillium dahliae. 1-octen-3-one showed the highest activity, followed by 2-octen-1-ol and 1-octen-3-ol, while 3-octanone had the lowest activity. Half maximum effective concentration (EC50) values were 14.59, 240.1, 432.9, and 3923 μL/L. The inhibitory activity of VOCs with different structures may be attributed to the higher activity of ketone groups than alcohol groups (11-alcohol), as well as differences in the positions of carbon-carbon double bonds (13-alcohol), and combinations of carbon-carbon double bonds and alcohol groups (E) and 11-alcohol). The inhibitory effect of 1-octen-3-ol was stronger than that of 3-octanone, which was consistent with the inhibition results against four other pathogenic fungi (Rhizoctonia solani, Fusarium oxysporum, Phytophthora infestans, and Phytophthora citrophthora). The addition of 1 μL of VOCs inhibited the spore germination of Verticillium dahliae. However, compared with other VOCs, the inhibitory effect of 3-octanone was relatively low.

2. Inhibitory Effect on Conidial Germination of Verticillium dahliae

A 200 μL culture system was established by adding 10 μL of methanol solution (including 1 μL of VOCs), 90 μL of PDB solution, and 100 μL of spore suspension (with a concentration of 1×10⁶ spores) into a 1.8 mL eppendorf (EP) tube. The culture was incubated at 150 rpm and 25° C. The inhibition rate was calculated as follows: inhibition rate (%)=[(spore germination rate of control group−spore germination rate of treatment group)/spore germination rate of control group]×100. The germination rates of the control group and treatment group were calculated at 24 h and 48 h under the same field of view.

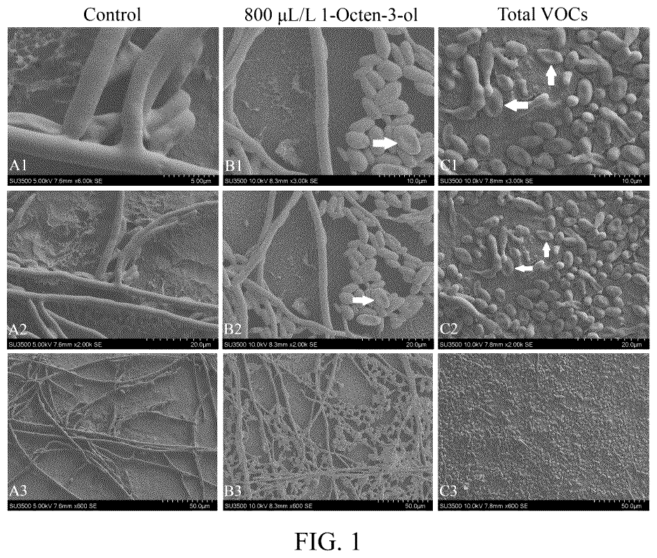

1-octen-3-one, a major VOC component of CEF642N, reduced mycelial development when treating Verticillium dahliae. One treatment was with 1-octen-3-one at a concentration of 800 μL/L. With the extension of treatment time, the inhibition rate gradually decreased. This indicates that 1-octen-3-one may also induce some responses in Verticillium dahliae, thereby reducing the inhibitory effect of external stress. In the inverted paired assay between CEF642N and Vd076, the total VOCs produced by CEF642N gradually increased with the extension of treatment time, and the inhibition rate of Verticillium dahliae also gradually increased. SEM images showed that the control group was mainly mycelia (FIG. 1A), while the 1-octen-3-one treatment group had partial mycelia and spores, with some spores shrinking (FIG. 1B). The VOC treatment group was included by spores, which also shrank (FIG. 1C), indicating that VOCs hinder spore germination. The total VOCs of CEF642N and 1-octen-3-one both had effects on spore germination and development, as well as mycelial growth of Verticillium dahliae.

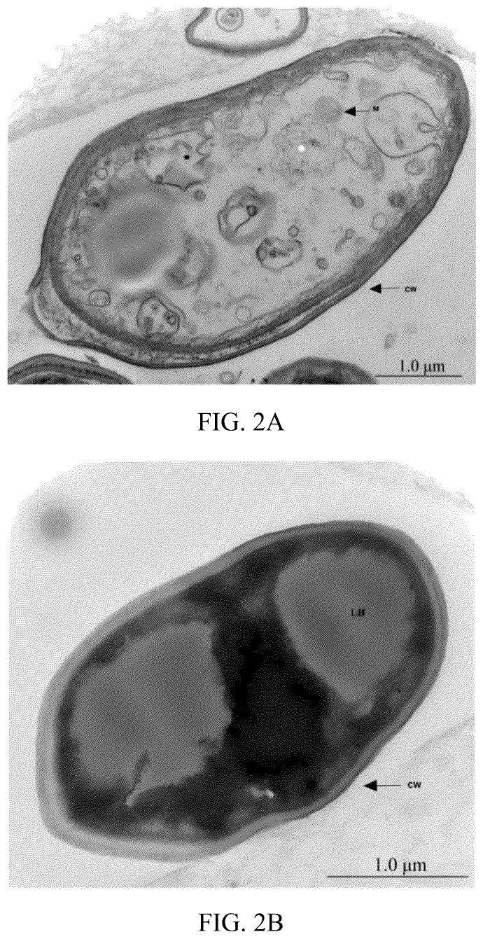

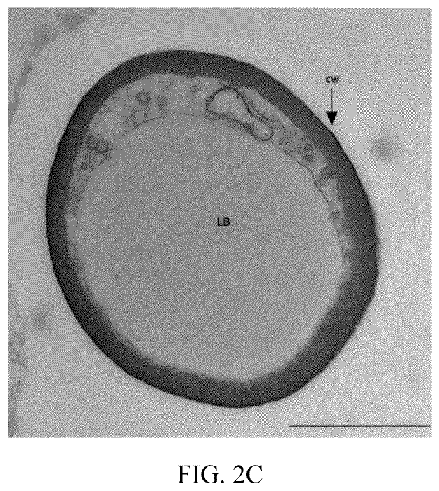

TEM observations showed that compared with the control group, 1-octen-3-one and total VOCs from CEF642N caused severe organelle damage, mitochondrial degeneration, and cavitation. In the study, VOCs reduced the mycelial growth, spore germination, and internal structure of pathogen cells of Verticillium dahliae (FIGS. 2A, 2B and 2C).

### Example 3: Hyperparasitic Effect of Talaromyces purpureogenus CEF642N on Verticillium dahliae Vd076

The time course of fungal parasitism was consistent with the description of the confrontation culture between CEF642N and Vd076 mentioned above. CEF642N and Vd076 (5 mm in diameter) were inoculated onto PDA medium. In addition, PDA media coated with Vd076 spores were cultured on three different media for 7 days. The colony diameter of CEF642N was measured and photographed every 3 days. For the confrontation culture of CEF642N and Vd076, the fungal parasitic parts of the confrontation culture were detected by SEM.

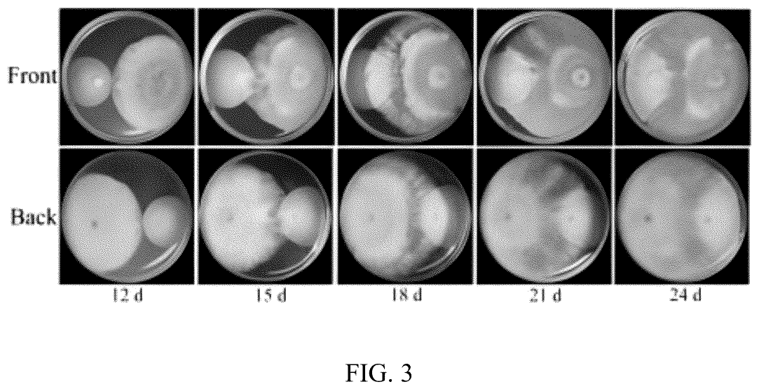

On the 12th day of confrontation culture, CEF642N and Vd076 came into contact. With the extension of co-culture time, the coverage area of CEF642N on Vd076 steadily expanded, indicating an increase in the fungal parasitism rate (FIG. 3). During the co-culture of CEF642N and Vd076, CEF642N may produce antibiotics to kill the pathogen and secrete cell wall-degrading enzymes, including chitinase and β-1,3-glucanase, leading to fungal parasitism.

When CEF642N was inoculated into the medium already inoculated with Verticillium dahliae, it could be observed that CEF642N might grow on Vd076 mycelia CEF642N may be on the medium covered with Vd076 spores. This indicates that CEF642N can perform biotrophic parasitic growth on Vd076, utilizing the nutrients of Verticillium dahliae for its own development. When comparing the colony diameters of CEF642N, it was evident that the PDA medium had the largest colony diameter, and there were significant differences in colony sizes between Vd076 mycelia and Vd076 spores. Biotrophic parasitic fungi use various mechanisms to collect nutrients from living hosts.

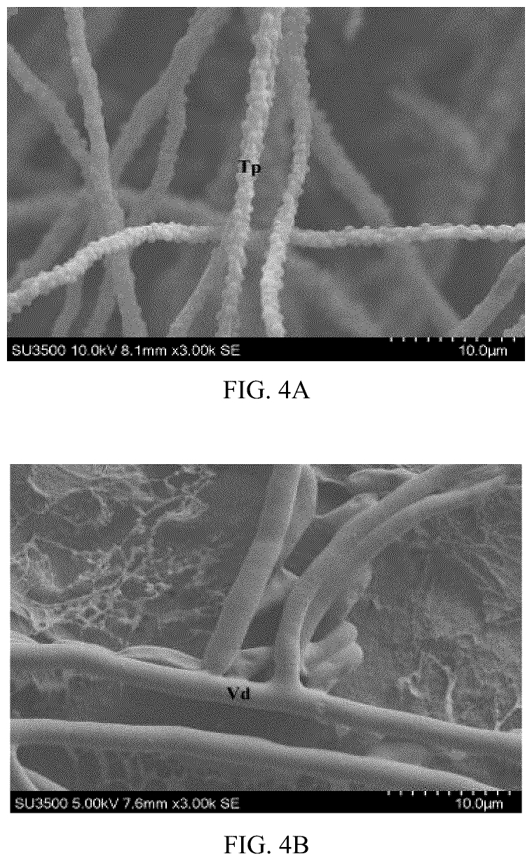

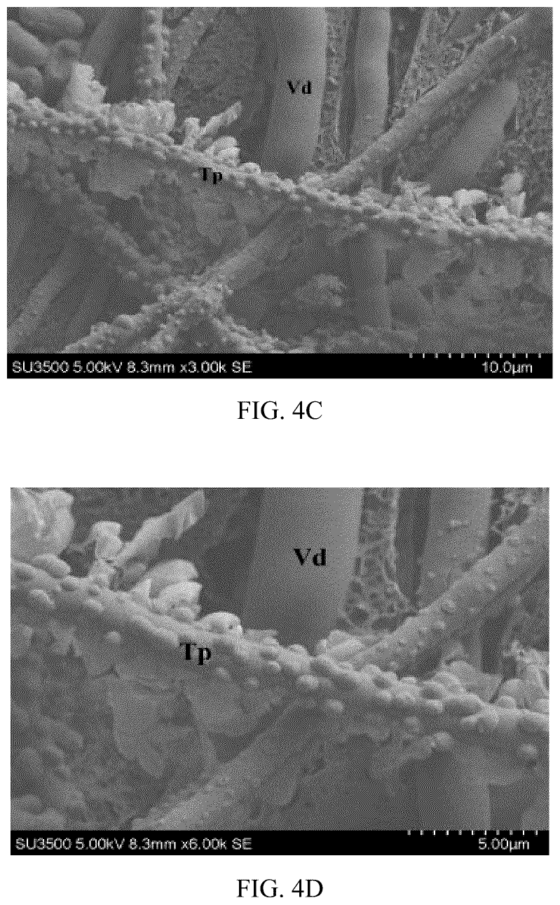

SEM images of CEF642N cultured alone on PDA medium (Tp) showed numerous protrusions on its mycelia (FIG. 4A), while Vd076 (Vd) mycelia were smooth (FIG. 4B). In the mycoparasitic part of the confrontation culture, CEF642N mycelia could grow parallel to Verticillium dahliae (FIGS. 4C and 4D).

In summary, the present disclosure screened the inhibitory effect of Talaromyces purpureogenus CEF642N on Verticillium dahliae. It can inhibit the growth of Verticillium dahliae through VOCs and non-volatile secondary metabolites, and also exert a fungal parasitic effect on Verticillium dahliae. Genome sequencing analysis showed that CEF642N includes a large number of lipid metabolism genes, which may be precursors of antagonistic VOCs, as well as genes related to secondary metabolites such as terpenoids and polyketides. Genome comparison analysis indicated that CEF642N was evolutionarily closer to Talaromyces stipitatus and had a more similar chemical phenotype. Carbohydrate-active enzymes may have more cell wall-degrading functions after gene deletion and neofunctionalization. The mechanism of action of CEF642N on Verticillium dahliae is that VOCs target the mitochondria and cell membrane of the pathogen in the early stage, followed by the action of non-volatile secondary metabolites, and finally the occurrence of parasitic effect after contact. The results show that CEF642N has multiple antagonistic effects and broad application prospects in the control of cotton Verticillium wilt.

1. A strain of Talaromyces purpureogenus, wherein a deposit number of the Talaromyces purpureogenus is CCTCCNO: M20242093.

2. An application of the Talaromyces purpureogenus according to claim 1 in preparing a biocontrol agent in control of cotton Verticillium wilt caused by Verticillium dahliae.

3. The application according to claim 2, wherein the biocontrol agent is a liquid formulation.

4. A method for controlling cotton Verticillium wilt, wherein the method uses the Talaromyces purpureogenus according to claim 1 to control cotton Verticillium wilt caused by Verticillium dahliae.
